(19) United States
(12) Patent Application Publication
(10) Pub. No.: US 2011/0284007 A1
(43) Pub. Date: Nov. 24, 2011

(54) POSITIVE PRESSURE DEVICE

(76) Inventor: Peron PIERRE, Brooklyn, NY (US)

(21) Appl. No.: 13/094,536

(22) Filed: Apr. 26, 2011

Related U.S. Application Data
(60) Provisional application No. 61/347,077, filed on May 21, 2010.

(51) Int. Cl. A61M 16/04

(52) U.S. Cl. 128/207.16

(57) ABSTRACT

A positive pressure endotracheal device used with an air source which provides transmission of continuous positive pressure into the lungs using an inspiratory line and a one-way valve placed within the inspiratory line. This one-way valve will not allow exhaled air to return through the inspiratory line. The pressure in the device is measured with a monitor line which is connected to the air source and the disclosed Positive Pressure Endotracheal Device. The device includes: 1) WYE molded endotracheal connector interface, 2) a primary pressure release port, 3) a pressure monitor port, 4) a dispenser port (optional), 5) and inspiratory line (smooth walled), 6) a one-way valve connected to the inspiratory line, 7) an expiratory line (smooth walled), and 8) a peep valve connected on an end of the expiratory line to control lung inflation pressure at the end of exhalation.

This application claims the benefit of prior filed U.S. Provisional application SN 61/347,077, filed May 21, 2010.

### FIELD OF THE INVENTION

The invention relates to the field of positive pressure breathing aide devices. Specifically the invention relates to a positive pressure endotracheal device.

### BACKGROUND OF THE INVENTION

Currently there are a number of solutions for applying positive pressure for individuals with periods of apnea, hypoxia, atelectasis, and increased work of breathing. Some of these solutions attempt to use conventional mechanics, but these solutions fail to meet new requirements, which are the economical, environmental, physiological factors for today's rapidly changing medical environment. The current and expected requirements are incorporated into the positive pressure endotracheal device disclosed herein which allows easy access and comfort with safe, consistent and easily operable connections.

### SUMMARY OF THE INVENTION

The present invention relates to a positive pressure endotracheal device used with an air source which provides transmission of continuous positive pressure into the lungs using an inspiratory line and a one-way valve placed within the inspiratory tube. This one-way valve will not allow pressure to return through the inspiratory line to the air source. The pressure is measured with a monitor line which is connected to the source and the Positive Pressure Endotracheal Device (PPETD). As insufflations occur the lungs expand creating an increasing pressure gradient. This lung pressure is maintained due to the presence of a cuffed (inflated around the placed endotracheal tube) inserted tube. Involuntary exhalation phase occurs as follows: The lung exhalation pressure follows the path of least resistance, which is through a second line exhalation tube. A positive end expiratory pressure (PEEP) valve placed inside the tube maintains lung expansion, at a predetermined adjustable pressure, while allowing the excess exhaled lung pressure release.

It would be desirable to have a device which when applied will improve oxygenation and decrease the work of breathing while, simultaneously maintaining positive end expiratory pressure (peep). Furthermore it is desirable to have a device which is environmentally safe. The positive pressure endotracheal device of the present invention utilizes air coming from a source, i.e., wall O2/O2 tank/air concentrator. Still further this device is cost efficient and will meet the need in the medical industry to have a tool to improve the lives of many individuals with poor oxygenation and lung complications. This endotracheal device will inflate the lungs and allow passive exhalation to atmospheric pressure while maintaining a sustained positive end expiratory pressure with the use of a peep valve.

The present invention facilitates lung recruitment with the use of the one-way valve, placed proximal in the inspiratory line to create a pressure differential, allowing the one-way valve to remain open. A peep valve placed distal in the expiratory line creates an increase pressure gradient within the lungs, while allowing passive exhalation in a controlled manner.

One embodiment of the present invention is made of the following components: 1) A WYE endotracheal connector, 2) a pressure release port, 3) a pressure monitor port, 4) a dispenser port, 5) an inspiratory line, 6)a one way valve, 7) a smooth walled expiratory line, and 8) a peep valve. The positive pressure endotracheal device entails a continuous transmission of positive pressure into the lungs of a spontaneously breathing patient using a cuffed endotracheal tube.

The present invention provides a method associated with the disclosed endotracheal device which includes the following assembly and administration steps. A WYE endotracheal connector is used to interface with the trachea, using a cuffed endotracheal tube. A pressure release port placed on the proximal end of the molded WYE interface is used to facilitate emergency pressure release. A pressure monitor port is positioned vertically on the molded WYE endotracheal connector to monitor lung pressure. An internal one-way valve prevents pressure returning into the inspiratory line. A smooth walled exhalation line is connected to the expiratory side of the WYE connector and a peep valve connected on a distal end thereof provides resistance during the exhalation phase, allowing predetermined exhaust lung volume.

The present invention is unique in that it is structurally different from other known devices or solutions. More specifically, the invention is unique due to the presence of a one-way valve placed inside or outside a single inspiratory line, which transmits and maintains a positive pressure into the lungs and facilitates lung expansion and alveoli recruitment when used with a cuffed endotracheal tube. A sustained recruitment is established. Exhaled lung volume is controlled with the use of a peep valve placed within the expiratory line, which will maintain alveoli recruitment and facilitate a predetermined lung exhaust.

### BRIEF DESCRIPTION OF THE DRAWINGS

FIG. 1 is a plan view of the device.

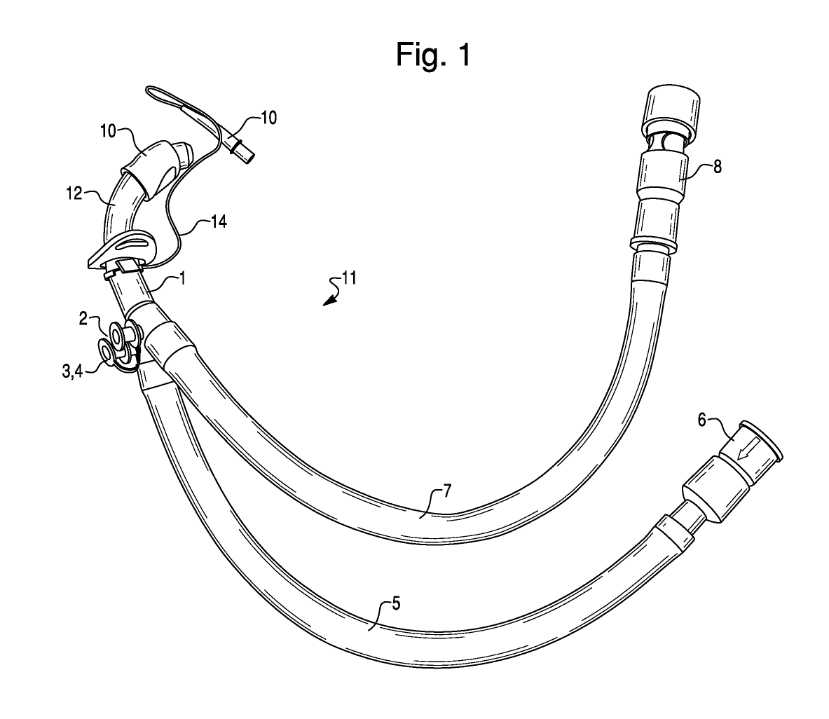

### DETAILED DESCRIPTION

The present invention is a positive pressure endotracheal device 11 which is made up of the following components: 1) WYE molded endotracheal connector interface suitably sized for the patient to be treated (i.e., adult, child, infant, etc.), 2) a primary pressure release port(associated with said WYE connector preferred), 3) a pressure monitor port (associated with said WYE connector preferred), 4) a dispenser port (associated with said WYE connector preferred), 5) and inspiratory line (smooth walled preferred), 6) a one-way valve, 7) an expiratory line (smooth walled preferred), and 8) a peep valve. A tracheal tube 12 is also shown with a deflated cuff 10 and associated inflator line 14. During use, the tracheal tube would be in-place within the patient.

The positive pressure provides a method associated with the endotracheal device which includes the following steps: 1) an endotraeheal WYE connector is oriented no as to facilitate interface with the endotracheal tube, 2) a pressure release port is placed on the proximal end of the molded tracheal interface, 3) a pressure monitor is also positioned, vertically, on the molded endotracheal connector, 4) a dispenser port is established, 5) an internal one-way valve is placed inside/outside the inspiratory limb of the assembly so as to prevent sourced air from passing backwards through the inspiratory line, 6) an expiratory limb (smooth walled) tubing is connected to the WYE, and 8) a peep valve placed at distal end of the expiratory limb or otherwise within the flow path of the expiratory limb.

In general the device operates as follows: air flows from a suitable source and passes through the tubing into the one-way valve 6 which will not allow air to return into the tubing 5 once air has passed through to the patient. The air flows through the molded endotracheal WYE interface 1 into the trachea thereby inflating the lungs. A cuffed 10 endotracheal tube 12 traps the air inside the lungs and prevents exhaled air passing around the cuff 10 and out through the patient nose or mouth. As the lung inflates, exhalation (passive) occurs forcing the exhaled lung volume out through the exhalation tubing 7. As the air follows the path of least resistance the peep valve 8 limits the amount of exhaled gas while allowing exhalation to occur.

In detailed operation the positive pressure endotracheal device works as follows: 1) the source flow/O2 tank/wall O2/provides intermittent/continuous positive pressure, 2) as flow enters the PPD inspiratory line 5, a pressure differential is created as the flow enters the one way valve 6, 3) the one-way valve 6 will remain open as flow remains constant, and the source air pressure exceeds the pressure within the inspiratory line. The one-way also will not allow exhaled exhaust to return through the tube 5; the inflation of the lungs occur, producing an increase pressure gradient, 4) elastic recoil, exhalation, occurs due to an increase pressure gradient, exhaled lung exhaust follows the path of least resistance, 5) through the expiratory line 7, this exhaled lung volume is limited by the use of a resistor, (peep) positive end expiratory pressure valve 8, and 6) this resistor (peep valve) will limit the exhaled lung volume to a predetermined volume allowing exhalation to a more physiological way of breathing.

While a specific version of this device has been disclosed, it will be understood that reasonable variation, such as nasal, tracheal, mask applications will, occur to one of ordinary skill in the field. This invention is intended to be limited only as by the claims below.

1. A positive pressure tracheal device, comprising:
an inspiratory line, for accepting positive pressure air therein, connected to a one-way valve that allows said air to flow into said inspiratory line in a single flow direction from a positive pressure air source;
a wye connector connected to an end of said inspiratory line and receiving positive air pressure from said inspiratory tine and conducting said air to a cuffed tracheal tube for introducing said air into a patient's airways;
an expiratory line connected to said wye connector for conducting exhaled air delivered at an exhalation pressure exceeding said inspiratory air from said patient to said wye connector; and,
said expiratory line including an adjustable positive end expiratory pressure valve for releasing air from said expiratory tine and for adjusting the lung inflation pressure of said patient using said device.

2. A device as in claim 1, further comprising:
a pressure release port on said wye connector.

3. A device as in claim 1, further comprising;
a pressure monitor port on said wye connector.

4. A device as in claim 1, further comprising:
a dispenser port on said wye connector.

5. A method of using a positive pressure device, comprising:
selecting a suitable wye connector for administration of supplied air o a particular patient,
connecting an inspiratory line to a branch of said wye connector and equipping said inspiratory line with a one-way valve for eliminating back airflow through said inspiratory limb,
connecting an expiratory line to said wye connector, said expiratory line being equipped with a peep valve so as to maintain a predetermined pressure within said expiratory line when said positive pressure device is administered to a patient,
connecting said wye connector to a cuffed endotracheal tube already positioned within said patient, and
connecting said inspiratory line to a source of air for administration of positive air pressure supplied through said one-way valve to said patient.
